(19) United States
(12) Patent Application Publication
(10) Pub. No.: US 2020/0297569 A1
(43) Pub. Date: Sep. 24, 2020

(54) COMPRESSION DEVICES

(71) Applicant: MAS Innovation (Private) Limited, Colombo 02 (LK)

(72) Inventors: Karunaratne ANGELO, Colombo (LK); Fernando KANISHKA, Colombo (LK); Perera JANMI, Colombo (LK); Karthikan THEIVENDRAN, Colombo (LK)

(21) Appl. No.: 16/649,457

(22) PCT Filed: Sep. 24, 2018
(86) PCT No.: PCT/SG2018/050484

§ 371 Date: Mar. 20, 2020

Related U.S. Application Data
(60) Provisional application No. 62/561,827, filed on Sep. 22, 2017.

(51) Int. Cl. A61H 1/00; G16H 40/63

(52) U.S. Cl. CPC A61H 1/006; G16H 40/63

(57) ABSTRACT

The present disclosure relates to compression devices for applying compression to a limb of a subject. Embodiments disclosed relate to compression patterns applied by a compression device configured to apply compression to a plurality of compression zones on a limb of a subject, synchronization signals sent from a master compression device to a slave compression device, and to the provision of baffles in an inflatable bladder of a compression device.

### FIELD OF THE INVENTION

The present disclosure relates to compression devices for applying compression to a limb of a subject. In particular, embodiments disclosed herein relate to actuators for compression devices for applying compression to different zones on a limb and to compression patterns applied by compression devices.

### BACKGROUND OF THE INVENTION

Arteries carry blood from the heart out to the rest of the body and veins carry blood back to the heart, and valves in the veins stop the blood from flowing backward. About 90% of venous return from the legs is through the action of the muscle pumps. The calf muscle pump is the most important muscle pump in the leg and is active during walking and ankle movement. As a result, the effectiveness of the calf muscle pump depends on normal calf muscle activity. This itself requires good ankle mobility, a normal gait and lack of neurological deficit.

Calf muscle function decreases with increasing age, at least partly as a result of reduced muscle bulk. In addition, when veins have trouble sending blood from the limbs to the heart venous insufficiency occurs. In this condition, the flow of blood through the veins is inadequate, causing blood to pool in the legs.

Active compression benefits people with lower limb circulation issues or at risk for blood clots in the legs by improving venous return through effective muscle pump action.

For athletes, adequate recovery has been shown to result in the restoration of physiological and psychological processes, so that the athletes can compete or train again at an appropriate level. Many recovery strategies for elite athletes are based on medical equipment or therapies used in patient populations. Compression clothing is one of these strategies that have been traditionally used to treat various lymphatic and circulatory conditions. Compression garments are thought to improve venous return through application of graduated compression to the limbs from distal to proximal. The external pressure created may reduce the intramuscular space available for swelling and promote stable alignment of muscle fibers, attenuating the inflammatory response and reducing muscle soreness.

Massage is a widely used recovery strategy among athletes. However, apart from perceived benefits of massage on muscle soreness, few reports have demonstrated positive effects on repeated exercise performance. Furthermore, increased blood flow is one of the main mechanisms proposed to improve recovery (thus improving clearance of metabolic waste products).

Active compression devices may also be beneficial for athletes by improving venous return through the application of active compression to the limbs from distal to proximal. The external pressure created reduces the intramuscular space available for swelling and promote stable alignment of muscle fibers, reducing the inflammatory response and muscle soreness. Also, the contraction and relaxation of the muscle groups and blood vessels will mimic the calf muscle pump activity, thereby increasing circulation and reducing the lactic acid build up on the muscles. This is beneficial in improving psychological aspects of recovery and may have potential benefits for injury prevention, management and improving performance.

### SUMMARY OF THE INVENTION

Some aspects of the present disclosure relate to compression patterns which are applied to the limb of the subject. The compression patterns generally comprise a sequence of compression cycles. Each compression cycle may comprise a compression period followed by a relaxation period. In the compression period compression pressure is applied to the limb of the subject. In the relaxation period, the compression pressure is reduced to a initial pressure.

According to first aspect of the present disclosure, a method of controlling a compression device is provided. The compression device is configured to apply compression to a plurality of compression zones on a limb of a subject. The compression zones are each at a different distance from the torso of the subject. The method comprises: in a compression period, generating control signals to separately vary the pressure applied in each of the compression zones such that for each adjacent pair of compression zones the pressure applied at a distal zone of the pair of compression zones is greater than to the pressure applied at a proximal zone of the pair of compression zones.

In some embodiments in at least part of the compression period, the pressure in each of the compression zones is varied simultaneously. The fact that the zones operate simultaneously, and sometimes with a few seconds between starting of the compression cycle, ensures that the subject's blood is continuously moving in the limb without any holding periods.

Many existing compression devices compress a leg zone by zone individually which makes the blood stagnant in one particular zone or compartment.

In some embodiments the method comprises generating control signals for a plurality of compression cycles, each compression cycle comprising a compression period followed by a relaxation period.

In some embodiments the method comprises generating control signals to reduce the pressure applied in each compression zone during the relaxation period.

In some embodiments, a first type of compression pattern is used wherein during the compression period, the pressure applied in each of the zones is gradually increased from a first pressure to a second pressure and compression of the distal zone of a pair of compression zones starts before the proximal zone of the pair of zones.

In some embodiments, a second type of compression pattern is used wherein the compression pressure applied to each compression zone is repeatedly varied between a maximum value for that zone and a minimum value for each zone. This pulsing sequence creates a vacuum in the veins of the subject causing more blood to be drawn in from lower zones which makes the compression therapy more effective.

In some embodiments, the method comprises reducing the compression pressure applied at a given zone within 5 seconds of the applied compression pressure reaching the maximum value for that zone. In some embodiments the method comprises reducing the compression pressure applied at a given zone within 2 seconds of the applied compression pressure reaching the maximum value for that zone.

If a zone is held at its maximum pressure for a more than a few seconds, the result is that there is no antegrade blood flow from that zone. Thus by starting the relaxation period within 5 seconds or less after a zone reaches its maximum pressure it is ensured that there is always a continuous blood flow in the veins.

In an embodiment during a compression period, the maximum value for the proximal zone of the pair of zones is substantially equal to the minimum value for the distal zone of the pair of zones. The difference between the maximum value and the minimum value for a zone is less than 30 mmHg and preferably less than 20 mm Hg in some embodiments.

An advantage of such embodiments is that during the compression period, the pressure only varies by a relatively small amount, thus energy consumption is reduced.

In some embodiments, the compression device is configured to send a synchronization signal to a second compression device. This provides for synchronization of compression patterns applied to each leg of a subject. The synchronization signal may be an infrared signal or a wireless network signal. The synchronization signal may comprise an indication of the compression pattern.

According to a second aspect of the present disclosure, there is provided a compression device which is configured to apply compression to a limb of a subject according to one of the compression patterns described above. The compression device may be operable to provide both patterns and may be provided with a user interface to allow selection of a desired compression pattern.

According to a third aspect of the present disclosure there is provided a computer readable carrier medium carrying instructions which are executable by a controller of a compression device to cause the device to apply compression to a limb of a subject according to one of the compression patterns described above. A user may be able to select a desired compression pattern from a plurality of options.

According to a fourth aspect of the present disclosure, there is provided a method of enhancing blood flow in a limb of a subject. The method comprises applying a compression device to a limb of a subject and controlling the compression device to apply compression according to one of the compression patterns described above.

According to a fifth aspect of the present disclosure there is provided a method of aiding recovery from physical exertion in a subject. The method comprises applying a compression device to a limb of a subject and controlling the compression device to apply compression according to one of the compression patterns described above.

A compression sleeve, such as a passive compression sock may be applied to the limb of the subject under the compression device.

According to a sixth aspect of the present disclosure, a method in a master compression device being configured to apply compression to a limb of a subject is provided. The method comprises receiving a user input indicating a selection of a compression sequence; generating control signals to apply the selected compression sequence to the limb of the subject; generating a synchronization signal for a slave compression device, the synchronization signal comprising an indication of the selected compression sequence; and transmitting the synchronization signal to the slave device.

According to a seventh aspect of the present disclosure a compression device configured to apply compression to a limb of a subject according to a compression pattern is provided. The compression device comprises a communication module configured to send a synchronization signal to a second compression device and/or receive a synchronization signal from a second compression device.

According to an eighth aspect of the present disclosure, a compression device comprising: a first inflatable bladder formed between a first membrane and a second membrane is provided. A plurality of baffles are provided that couple the first membrane to the second membrane.

The compression device may comprise a second inflatable bladder formed between the first membrane and the second membrane, wherein the first bladder and the second bladder are arranged to apply compression to compression zones each being a different distance from the torso of the subject. The first bladder and the second bladder may be configured to be independently inflated/deflated.

In some embodiments, the first bladder and the second bladder are separated by a boundary formed by bonding the first membrane to the second membrane.

In some embodiments the baffles are arranged parallel to an axis of the limb of the subject when the compression device is in use. Gaps may be provided in the baffles and the positioning of the gaps is offset between neighbouring baffles.

According to a further aspect of the present invention an actuator module for a compression device is provided. The actuator comprises a body portion and a pair of coupling portions. The body portion comprises an actuator configured to urge the coupling portions towards the body portion. The coupling portions are attached or attachable to an active layer which is configured to apply a compression pressure to a limb of a subject when the coupling portions are drawn towards the body portion.

In an embodiment, wherein the coupling portions are removably attachable to the active layer. The coupling portions are removably attachable to different locations on the active layer. This allows for adjustment to be made of the fit of the compression device to the subject. This may also allow the active layer to be pretensioned.

The actuator module may comprise a motor and the motor is coupled to a cable and pulley system configured to urge the coupling portions towards the body portion.

An actuator portion for a compression device may be formed from a plurality of actuator modules such that the coupling portions of each of the actuator modules is attached or attachable to different locations on the active layer such that different compression pressures can be applied to different compression zones of the limb of the subject.

### BRIEF DESCRIPTION OF THE DRAWINGS

In the following, embodiments of the present invention will be described as non-limiting examples with reference to the accompanying drawings in which:

FIG. 1 shows a compression device according to an embodiment of the present invention;

FIGS. 2a and 2b respectively show an active layer and an actuator portion of a compression device according to an embodiment of the present invention;

FIGS. 3a and 3b show the internal arrangement of an actuator component of a compression device according to an embodiment of the present invention;

FIG. 4 shows an actuator module which incorporates a controller and a battery according to an embodiment of the present invention;

FIGS. 5a and 5b show an actuator portion of a compression device according to an embodiment of the present invention;

FIG. 6 is a block diagram that schematically shows an actuator component of a compression device according to an embodiment of the present invention;

FIGS. 7a to 7i show variations of a first compression pattern type according to an embodiment of the present invention;

FIGS. 8a to 8f show variations of a second compression pattern type according to an embodiment of the present invention;

FIGS. 9a to 9d are graphs showing results of a sports study using a compression device according to an embodiment of the present invention;

FIGS. 10a and 10b are graphs showing results of a travel study using a compression device according to an embodiment of the present invention;

FIG. 11 shows schematically a pneumatic compression device according to an embodiment of the invention

FIGS. 12a to 12f show a pneumatic compression device according to an embodiment of the present invention;

FIG. 13 shows the internal configuration of an actuator portion of a pneumatic compression device according to an embodiment of the present invention;

FIG. 14 shows an inflatable bladder component within a pneumatic compression device according to an embodiment of the present invention;

FIG. 15 shows an inflatable bladder module of a compression device according to an embodiment of the present invention;

FIG. 16 shows a pair of compression devices which are provided with infrared communication modules according to an embodiment of the present invention; and

FIG. 17 shows a pair of compression devices which are provided with wireless communication modules.

### DETAILED DESCRIPTION

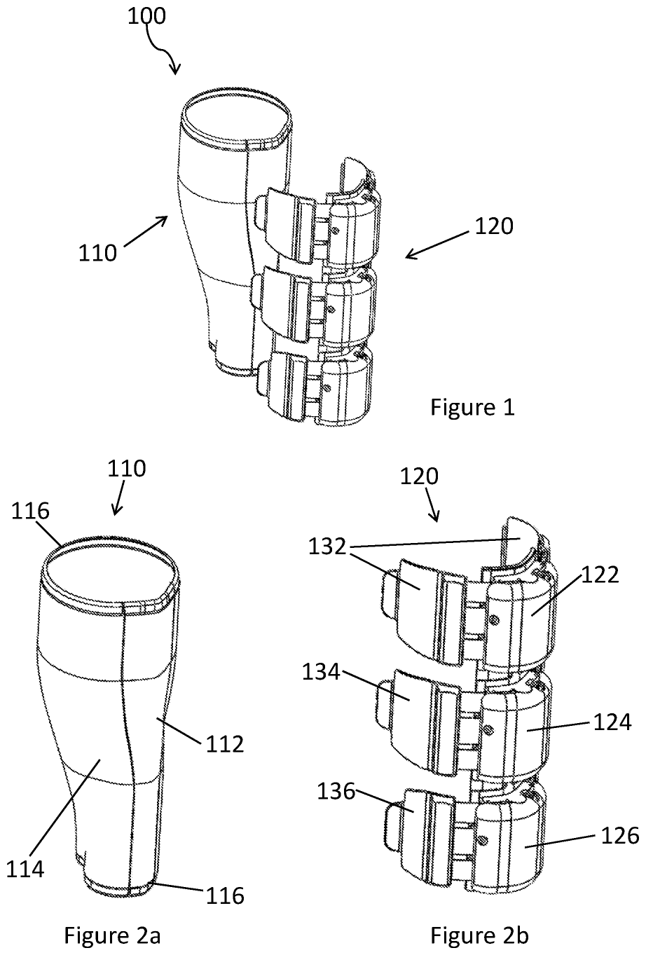

FIG. 1 shows a compression device according to an embodiment of the present invention. The compression device 100 comprises an active layer 110 and an actuator portion 120. The active layer 110 forms a compression sleeve or sock, which is worn on a limb of a subject. In this example, the active layer 110 is shaped to be worn on the lower leg of a human subject.

The actuator portion 120 is detachable from the active layer 110. When the actuator portion 120 is attached to active layer 110, the compression device 100 is operable to apply varying compression to different zones of the limb of the subject.

FIGS. 2a and 2b respectively show an active layer and an actuator portion of a compression device according to an embodiment of the present invention.

As shown in FIG. 2a, the active layer 110 is formed from a fabric material. In this example, the active layer 110 comprises a stretchable material part 112 that is located at the front of the wearer's leg and is located over the wear's shin when the active layer is worn. The active layer 110 further comprises a coupling portion 114 which allows actuator modules of the actuator portion to be attached to the active layer 110 and thereby apply compression to the limb of a subject. In this example, the coupling portion 114 of the active layer 110 is provided as a loop material provided on the exterior of the sides of the compression sleeve. This loop material can be detachably coupled to a hook material provided on arms of the actuator portion 120. The active layer 110 has a non-stretchable fabric part that is located at the sides and back of the wearer's leg when the active layer 110 is worn. The top and bottom edges of the active layer 110 are provided with an elastic edge binding 116.

In the embodiment described above in relation to FIG. 2A, the stretchable material part 112 serves two purposes. Firstly, it provides for adjustability to allow the active layer 110 fit closely to the limb of the subject. Secondly, the force provided by the stretchable material part 112 provides a base level of compression. In the following description this base level of pressure is called pretension pressure or initial pressure. It is envisaged that in embodiments, these attributes may be provided by alternative features, such as an adjustable strap and fastener which would allow the subject to manually vary the base level of compression and also to fit the active layer 110 to their limb. In some embodiments, a compression sock or sleeve may be worn under the active layer 110 to provide a base level of compression.

The non-stretchable part of the active layer 110 allows the transfer of mechanical energy generated by the actuator portion 120 to the leg muscles as a form of a compression without significant energy loss.

The materials of the active layer 110 may be selected to provide a smooth surface finish of the active layer 110 reducing friction between contact surfaces. Furthermore, fabrics with breathability, and moisture-wicking may be used.

As shown in FIG. 2b, the actuator portion 120 comprises three actuator modules 122, 124 and 126. A top actuator module 122 corresponds to an upper calf region, a middle actuator module 124 corresponds to a lower-calf region and a bottom actuator module 126 corresponds to an ankle region.

In this embodiment, a pair of arms 132 134 and 136 extends from each of the actuator modules 122 124 and 126. The inner surface of the arms comprises a hook material which can attach to the loop material on the active layer 110. Thereby a high shear resistant connection can be formed between the active layer 110 and the arms of the actuator modules. The arms 132 134 and 136 provide coupling portions of the respective actuator modules which are detachably couplable to the coupling portion 114 of the active layer 110.

It is envisaged that alternative methods of coupling the actuator modules 122 124 and 126 to the active layer may be used. For example, the arms 132 134 and 136 may be coupled to the active layer 110 via a hook and eye system, magnetic strips or via snap buttons. In alternative embodiments, the arms 132 134 and 136 may be replaced by straps which attach to the active layer 110 via buckles or other types of strap fasteners. Alternatively, cable loops may extend from the actuator modules and the active layer 110 may be provided with attachments for these cable loops.

Thus, the actuator portion 120 can be attached to the active layer 110 via respective coupling portions on the top actuator module 122, the middle actuator module 124 the bottom actuator module 126.

Each of the top actuator module 122, the middle actuator module 124 and the bottom actuator module 126 comprises a motor which is configured to pull the arms or more generally the coupling portion towards the actuator modules and thus increase the tension in the active layer 110. Thus the actuator portion 120 when attached to the active layer 110 operates to selectively increase the tension in different regions of the active layer 110 and thereby apply or modify compression pressure applied to different zones of the subject's leg or limb.

In the embodiment shown in FIGS. 1, 2a and 2b, three zones are defined by the actuator module. Thus, the top actuator module 122 defines a top compression zone, the middle actuator module 124 defines a middle compression zone and the bottom actuator module 126 defines a bottom compression zone.

Each of the actuator modules can be separately controlled to apply compression patterns to the subject's limb. These compression patterns are described in more detail below.

It is envisaged that actuators within the actuator modules 122 124 and 126 may be implemented with various actuation types. For example, pneumatic actuators which apply compression using a pump, or a compressed air or gas source which may be controlled by an electric current or voltage; electric actuators such as electric motors which convert electrical energy from a battery or other power source into mechanical energy; shape memory alloy actuators; or electro-active polymer based actuators may be used to implement the actuator modules. Examples of shape memory alloy based actuators are provided in PCT application publication WO2015038599; US patent application publication US20150073318; US patent application publication US20160374886; and U.S. Pat. No. 9,161,878. An example of an electro-active polymer material is provided in U.S. Pat. No. 9,433,537.

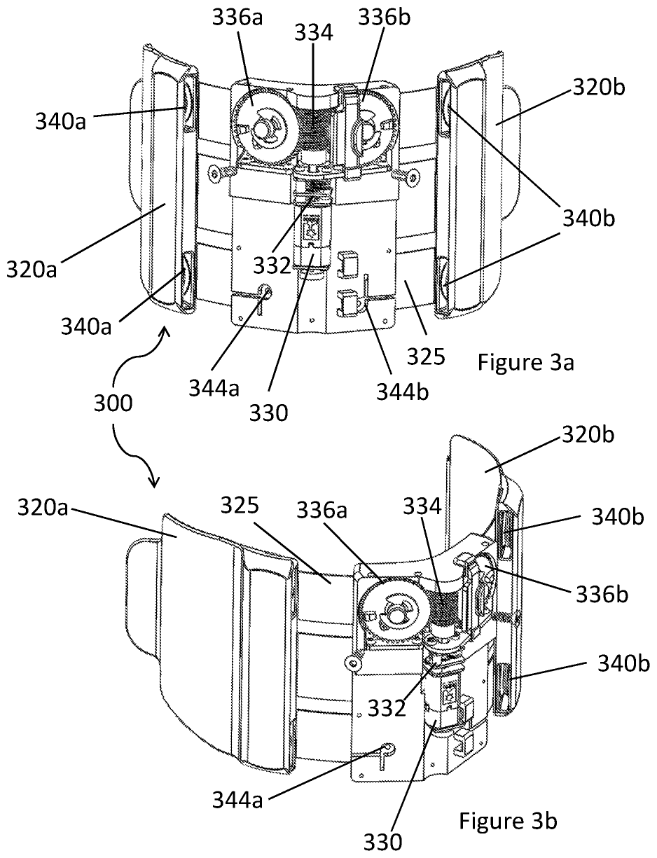

FIGS. 3a and 3b show the internal arrangement of an actuator component of a compression device according to an embodiment of the present invention. In this example, the actuation is provided by an electric motor. The actuator component 300 comprises a body portion 300 and two arm portions 320a and 320b. The arms 320a and 320b are mounted on sliders 325 which allow the arms 320a and 320b to move relative to the body portion 300.

The body portion 300 forms a housing for a motor 330, a gear box 332, and a worm gear set 334. The each of the cam gears 336a and 336b is coupled to a respective cable and pulley system which acts to pull the arm portions 320a and 320b towards the body portion 300.

A first cable (not shown in FIGS. 3a and 3b) runs from a spindle beneath one of the cam gears 336a through to pulleys 340a mounted on an arm 320a and back to an anchor point 344a on the body portion 300. Similarly, a second cable (also not shown in FIGS. 3a and 3b) runs from a spindle beneath the other of the cam gears 336b through to pulleys 340b mounted on an arm 320b and back to an anchor point 344b on the body portion 300. The arms 320a and 320b each have a travel relative to the body portion in the range 15 mm to 30 mm.

Compression is applied to a limb of the subject by the cables wound around the respective spindles and thus producing a force urging the arms towards the body of the actuator module. Thus causing the motors to rotate in one direction causes the compression pressure applied at a given zone to increase. Similarly, when the motors rotate in the opposite direction, the cable is released and the compression pressure applied to the limb of the subject reduces. In some embodiments, the actuator portion may be provided with a spring or similar mechanism to urge the arms outwards when the cable is released. In other embodiments, this outward force may be provided by either a stretchable portion of the active layer or simply by the release of compression pressure on the limb of the subject.

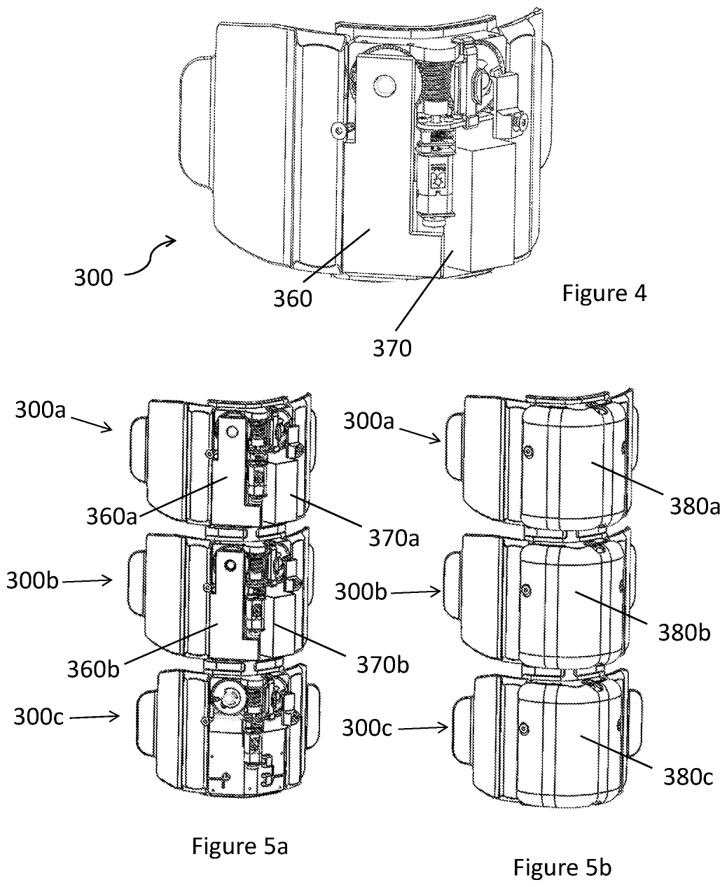

FIG. 4 shows an actuator module which incorporates a controller and a battery according to an embodiment of the present invention. As shown in FIG. 4, a controller module 360 in the form of a printed circuit board is mounted on one side of the actuator 300 and a battery is mounted on the opposite side of the actuator 300.

FIGS. 5a and 5b show an actuator portion of a compression device according to an embodiment. As shown in FIG. 5a, the top actuator module 300a and the middle actuator module 300b each comprise a respective controller module 360a and 360b; and a respective battery 370a and 370b in this example, the bottom actuator module 300c does not include a controller module and battery. It is noted that the control modules 370a and 370b may each contain parts of a complete controller which function together to control all three of the actuator modules. Similarly, the two batteries 370a and 370b may be coupled to the three actuators which in this example are motors. Thus the two controller modules 370a and 370b may be considered together as a single controller. Similarly, the two batteries 370a and 370b may be considered together as a single battery. In alternative embodiments the actuator modules may each comprise a separate controller and battery.

As shown in FIG. 5b, each of the respective actuator modules 300a 300b and 300c has a cover 380a 380b and 380c which protects the internal components of the actuator module. In an alternative embodiment, the three actuators may be covered by a single enclosure.

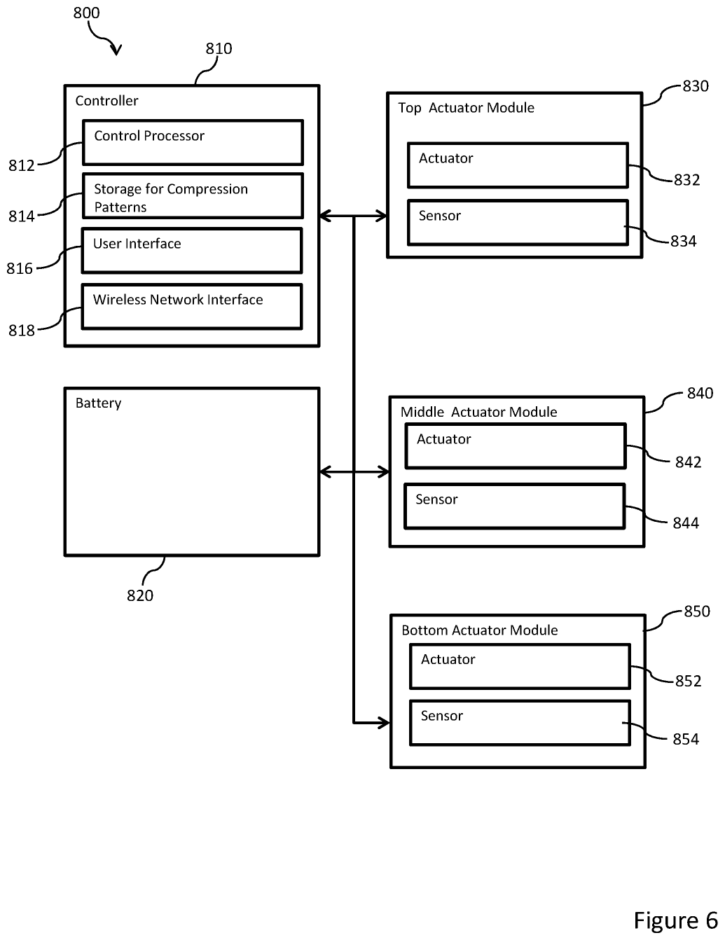

FIG. 6 is a block diagram that schematically shows an actuator component of a compression device according to an embodiment of the present invention. The actuator component 800 comprises a controller 810, a battery 820, a top actuator module 830, a middle actuator module 840, and a bottom actuator module 850.

The controller 810 comprises a control processor 812, storage for compression patterns 814, a user interface 816 and a wireless network interface 818. The control processor 812 may be implemented as a general-purpose processor that is operable to execute processor executable instructions or may be a hard-wired control processor. The storage for compression patterns 814 stores data that indicates compression patterns that may be used by the control processor 812 to generate control signals for motors in the actuator modules. The storage for compression patterns 814 may be implemented as a non-volatile storage. The user interface 816 may be implemented as any form of interface that allows a user to input commands and parameters. For example, the user interface may be implemented as a display and a plurality of input buttons, or a touch screen display. The wireless network interface 818 is an interface such as a Wi-Fi or Bluetooth interface which allows the controller to send and receive data and commands from a wireless enabled device such as a smartphone device or a computer device.

The battery 820 may be a rechargeable battery or a primary battery and may be implemented as a plurality of cells. The battery 820 supplies electrical power to the controller and the components of the actuator modules. In some embodiments, the battery may be replaced by a power supply configured to provide suitable electrical power, for example by converting a mains voltage.

As described above in relation to FIG. 5a, the battery 820 and controller 810 may be distributed among the actuator modules, alternatively, they may be housed in a separate control module.

The top actuator module 830 comprises an actuator 832 and a sensor 834. As described above, the actuator 832 is configured to apply a compression pressure to a top compression zone of a subject's limb. The sensor 834 is arranged on the compression device to measure the compression pressure applied to the top compression zone.

The middle actuator module 840 comprises an actuator 842, and a sensor 844. As described above, the actuator 842 is configured to apply a compression pressure to a middle compression zone of a subject's limb. The sensor 844 is arranged on the compression device to measure the compression pressure applied to the middle compression zone.

The bottom actuator module 850 comprises an actuator 852, and a sensor 854. As described above, the actuator 852 is configured to apply a compression pressure to a bottom compression zone of a subject's limb. The sensor 854 is arranged on the compression device to measure the compression pressure applied to the bottom compression zone.

The sensors 834 844 and 854 may be located on the respective actuator modules or may be located on the arms which couple to an active layer 110. The sensors 834 844 and 854 may be implemented as pressure sensors which directly measure the pressure applied to one of the respective compression zones, alternatively the sensors may be configured to indirectly measure a quantity from which the applied pressure can be derived or estimated, for example the sensors may be implemented as strain gauges mounted on the body of the actuator device and configured to measure the tension in the cables. In some embodiments the sensors may be implemented to measure variables of the actuator, such as the current or rotational in a motor position from which the torque on the motors and therefore the pressure applied to the compression zones can be derived. When the actuators are pneumatic the sensors may be air pressure sensors. When the actuators are shape memory alloys, the sensors may be temperature sensors.

Generally, the sensors will be configured to measure or sense the compression force or pressure. This measurement may be carried out by sensing variables of the actuators, such as input current, and deriving the compression pressure from these variables, or the compression force or compression pressure may be measured by the sensors.

When in operation, the control processor 812 generates control signals for the actuators of the actuator modules based on compression pattern data stored in the storage for compression patterns 814. These control signals may be, for example, controlled current and voltage signals to drive the respective motors. The control processor 812 may receive feedback signals from sensors such quadrature encoders and may adjust the current and voltage supplied to the actuators such that the pressure applied to the compression zones is as specified by compression pattern data stored in the storage for compression patterns.

While FIG. 6 shows the controller 810, the battery 820, the top actuator module 830, the middle actuator module 840 and the bottom actuator module 850 are separate modules, this is for illustrative purposes to simplify the functionality and it should be appreciated that the various functionalities may be housed in combination. For example, the functionality of the controller 810 and the battery 820 may be included in the housing of one or more of the actuator modules. The battery 820 may be implemented as a plurality of cells which may be split between housings of different actuator modules. In some embodiments the actuator modules may all be contained in a single housing with the controller 810 and battery 820 included in this housing.

It is also envisaged that some of the control processing implemented by the controller 810 by be carried out by a smart phone or other computing device which is coupled to the controller via the wireless interface 818. In such embodiments, the storage for compression patterns 814 may also be implemented in the smart phone or other computing device. It will also be appreciated that additional compression patterns may be downloaded and stored in the storage for compression patterns 814.

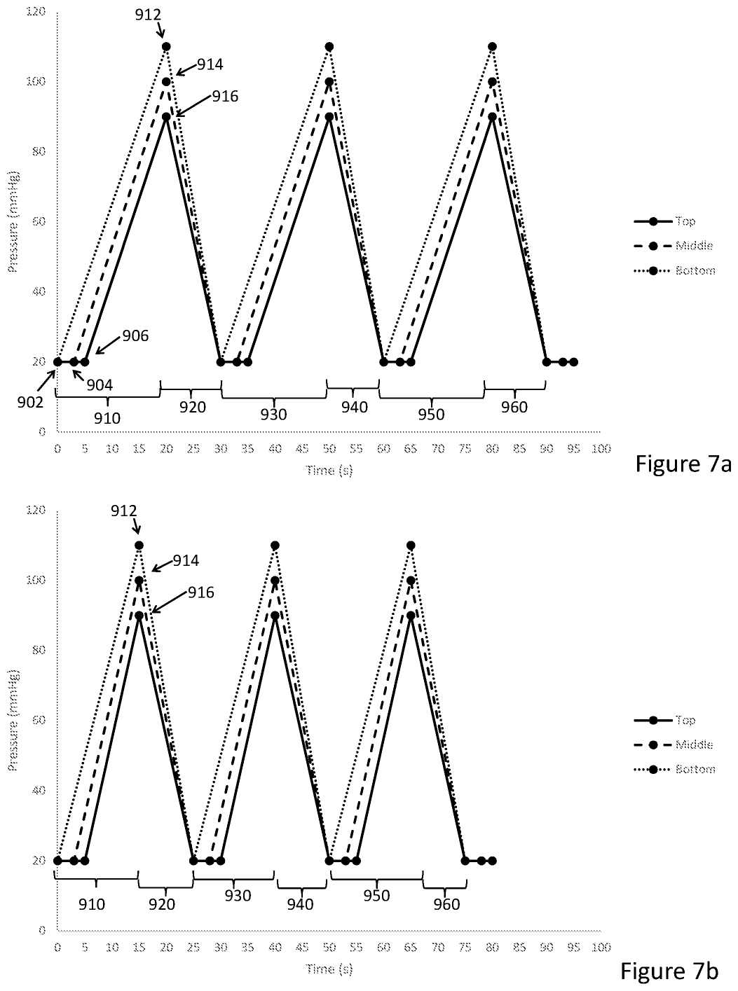

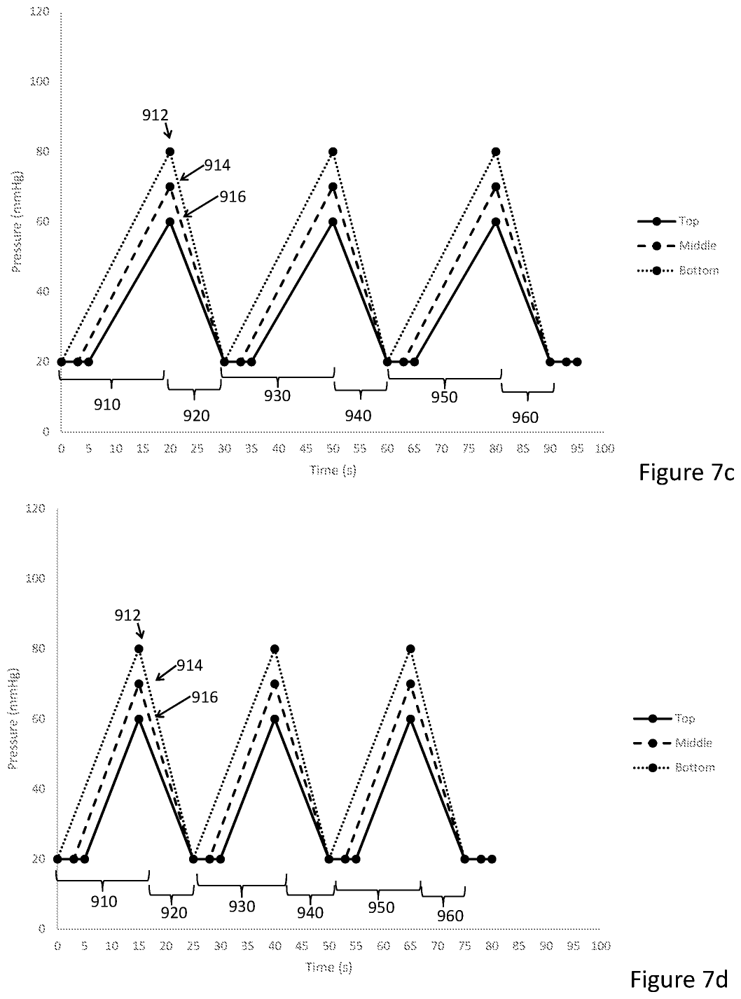

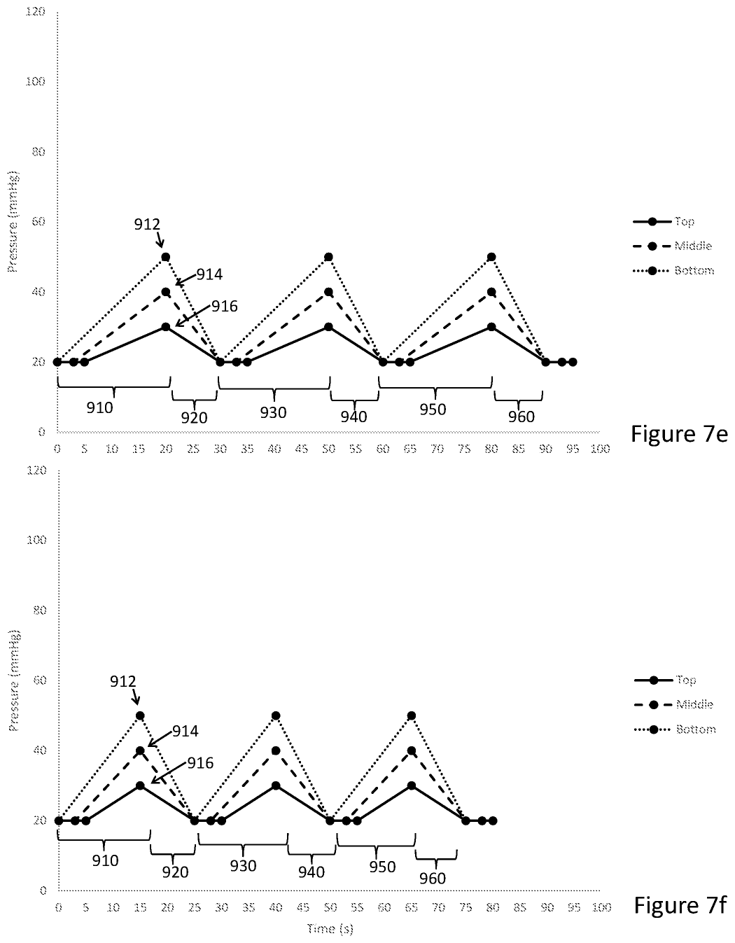

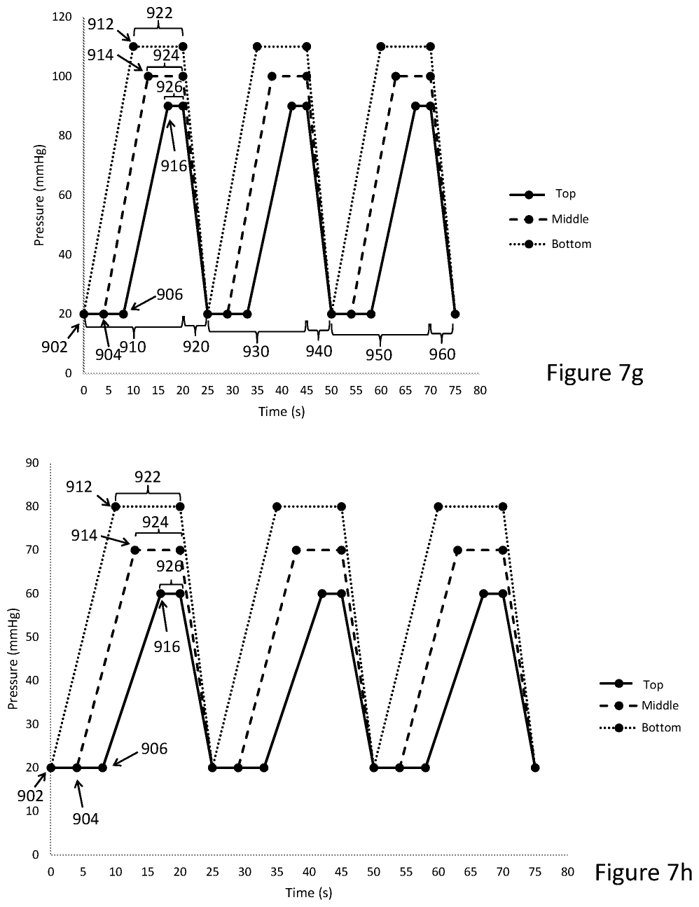

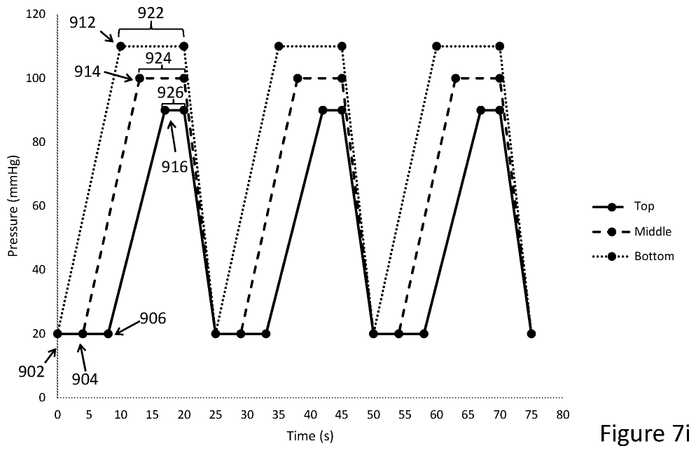

FIGS. 7a-i and 8a-f below show examples of compression patterns which may be stored in the storage for compression patterns 814.

FIGS. 7a to 7i show variations of a first compression pattern type according to an embodiment of the present invention.

The first compression pattern type involves a sequence of compression cycles. Each compression cycle may be considered to comprise a compression period followed by a relaxation period. During the compression period, the compression pressure in each of the compression zones is increased from an initial pressure or pretension pressure to a peak pressure value. During the relaxation period, the pressure in each of the compression zones returns to the initial pressure pressure.

Initially all of the compression zones are set to the initial pressure. This initial step may be implemented by the control processor 812 receiving feedback data from the sensors and controlling the actuators such that the pressure is adjusted to the initial pressure. Alternatively, the user may set the initial pressure by adjusting active layer 110.

In the Figures, the initial pressure is shown as 20 mmHg, however, this may be adjusted, either by the user as mentioned above, or to pre-programmed set values stored in the controller 810. In some embodiments, the initial pressure is set at a value in the range 10 to 30 mmHg for each of the compression zones. In other embodiments, different initial pressures or pretension pressures are set for different zones, for example the bottom zone may be set to an initial pressure of 110 mmHg, the middle zone to a initial pressure of 100 mmHg and the top zone may be set to an initial pressure of 90 mmHg.

Then, as shown in FIG. 7a, at a bottom zone start point 902, the compression pressure of the bottom zone is increased gradually from the initial pressure to a bottom zone peak value 912. Starting at a later time, the middle zone start point 904, the compression pressure of the middle zone is gradually increased from the initial pressure to a middle zone peak value 914. The middle zone peak value 914 is lower than the bottom zone peak value 912. Starting at a later time, the top zone start point 906, the compression pressure of the top zone is gradually increased from the initial pressure to a top zone peak value 916. The top zone peak value 916 is lower than the middle zone peak value 914.

The bottom zone reaches the bottom zone peak value 912 at approximately the same time as the middle zone reaches the middle zone peak value 914 and the top zone reaches the top zone peak value 916. The period between the bottom zone compression starting and all three zones reaching their respective peak values is referred to as a compression period 910.

Following the compression cycle 910, the compression pressure of the three zones is gradually reduced to the initial pressure. This reduction is referred to as a relaxation period 920. Once the three zones have returned to the initial pressure, a second compression period 930 begins and this is followed by a second relaxation period 940. A third compression period 950 and relaxation period 960 are then carried out.

In the embodiment shown in FIG. 7a, the bottom zone start point 902 is at 0 seconds, the middle zone start point 904 is at 3 seconds and the top zone start point 906 is at 5 seconds; the bottom zone peak value 912 is 110 mmHg, the middle zone peak value 914 is 100 mmHg and the top zone peak value 916 is 90 mmHg. The compression periods 910, 930 and 950 each last 20 seconds and the relaxation periods each last 10 seconds.

In the embodiment shown in FIG. 7b, the bottom zone peak value 912 is 110 mmHg, the middle zone peak value 914 is 100 mmHg and the top zone peak value 916 is 90 mmHg. The compression periods 910, 930 and 950 each last 15 seconds and the relaxation periods each last 10 seconds.

In the embodiment shown in FIG. 7c, the bottom zone peak value 912 is 80 mmHg, the middle zone peak value 914 is 70 mmHg and the top zone peak value 916 is 60 mmHg. The compression periods 910, 930 and 950 each last 20 seconds and the relaxation periods each last 10 seconds.

In the embodiment shown in FIG. 7d, the bottom zone peak value 912 is 80 mmHg, the middle zone peak value 914 is 70 mmHg and the top zone peak value 916 is 60 mmHg. The compression periods 910, 930 and 950 each last 15 seconds and the relaxation periods each last 10 seconds.

In the embodiment shown in FIG. 7e, the bottom zone peak value 912 is 50 mmHg, the middle zone peak value 914 is 40 mmHg and the top zone peak value 916 is 30 mmHg. The compression periods 910, 930 and 950 each last 20 seconds and the relaxation periods each last 10 seconds.

In the embodiment shown in FIG. 7f, the bottom zone peak value 912 is 50 mmHg, the middle zone peak value 914 is 40 mmHg and the top zone peak value 916 is 30 mmHg. The compression periods 910, 930 and 950 each last 15 seconds and the relaxation periods each last 10 seconds.

FIGS. 7g to 7i show a variation on the embodiments described above. As shown in FIG. 7g, at the bottom zone start point 902, the compression pressure of the bottom zone is increased gradually from the initial pressure to a bottom zone peak value 912. Starting at a later time, the middle zone start point 904, the compression pressure of the middle zone is gradually increased from the initial pressure to a middle zone peak value 914. The middle zone peak value 914 is lower than the bottom zone peak value 912. Starting at a later time, the top zone start point 906, the compression pressure of the top zone is gradually increased from the initial pressure to a top zone peak value 916. The top zone peak value 916 is lower than the middle zone peak value 914.

In this embodiment, the bottom zone reaches the bottom zone peak value 912 at earlier than the middle zone reaches the middle zone peak value 914 which is earlier than the time the top zone reaches the top zone peak value 916.

In this embodiment, the bottom zone is maintained at the bottom zone peak value 912 for a bottom zone peak period 922, the middle zone is maintained at the middle zone peak value 912 for a middle zone peak period 922 and the top zone is maintained at the top zone peak value 914 for a top zone peak period 924. The compression period for this embodiment includes the bottom zone peak period 924 and period between the bottom zone compression starting and the bottom zone reaching the bottom zone peak value 912 is referred to as a compression period 910.

Following the compression cycle 910, the compression pressure of the three zones is gradually reduced to the initial pressure. This reduction is referred to as a relaxation period 920. Once the three zones have returned to the initial pressure, a second compression period 930 begins and this is followed by a second relaxation period 940. A third compression period 950 and relaxation period 960 are then carried out.

In the embodiment shown in FIG. 7g, the bottom zone start point 902 is at 0 seconds, the middle zone start point 904 is at 5 seconds and the top zone start point 906 is at 10 seconds; the bottom zone peak value 912 is 110 mmHg, the middle zone peak value 914 is 100 mmHg and the top zone peak value 916 is 90 mmHg. The bottom zone peak period 922 is 12 seconds, the middle zone peak period 924 is 8 seconds and the top zone peak period 926 is 5 seconds. The compression periods 910, 930 and 950 each last 20 seconds and the relaxation periods each last 5 seconds.

In the embodiment shown in FIG. 7h, the bottom zone start point 902 is at 0 seconds, the middle zone start point 904 is at 3 seconds and the top zone start point 906 is at 5 seconds; the bottom zone peak value 912 is 80 mmHg, the middle zone peak value 914 is 70 mmHg and the top zone peak value 916 is 60 mmHg. The bottom zone peak period 922 is 10 seconds, the middle zone peak period 924 is 8 seconds and the top zone peak period 926 is 5 seconds. The compression periods 910, 930 and 950 each last 20 seconds and the relaxation periods each last 5 seconds.

In the embodiment shown in FIG. 7i, the bottom zone start point 902 is at 0 seconds, the middle zone start point 904 is at 3 seconds and the top zone start point 906 is at 5 seconds; the bottom zone peak value 912 is 110 mmHg, the middle zone peak value 914 is 100 mmHg and the top zone peak value 916 is 90 mmHg. The bottom zone peak period 922 is 10 seconds, the middle zone peak period 924 is 8 seconds and the top zone peak period 926 is 5 seconds. The compression periods 910, 930 and 950 each last 20 seconds and the relaxation periods each last 5 seconds.

In the embodiments described above, it is noted that during the compression cycles which may be considered to be formed from a compression period followed by a relaxation period, apart from when the zones are at the initial pressure, at any given time, the compression pressure applied to the bottom zone is greater than the compression pressure applied to the middle zone which in turn is greater than the compression pressure applied to the top zone.

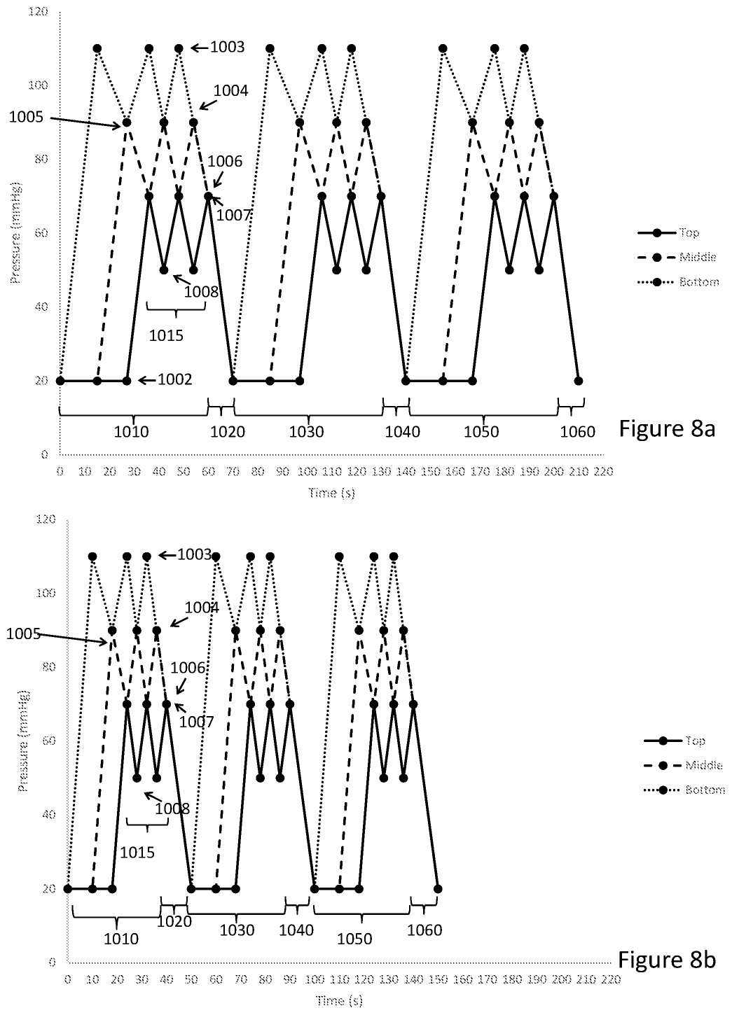

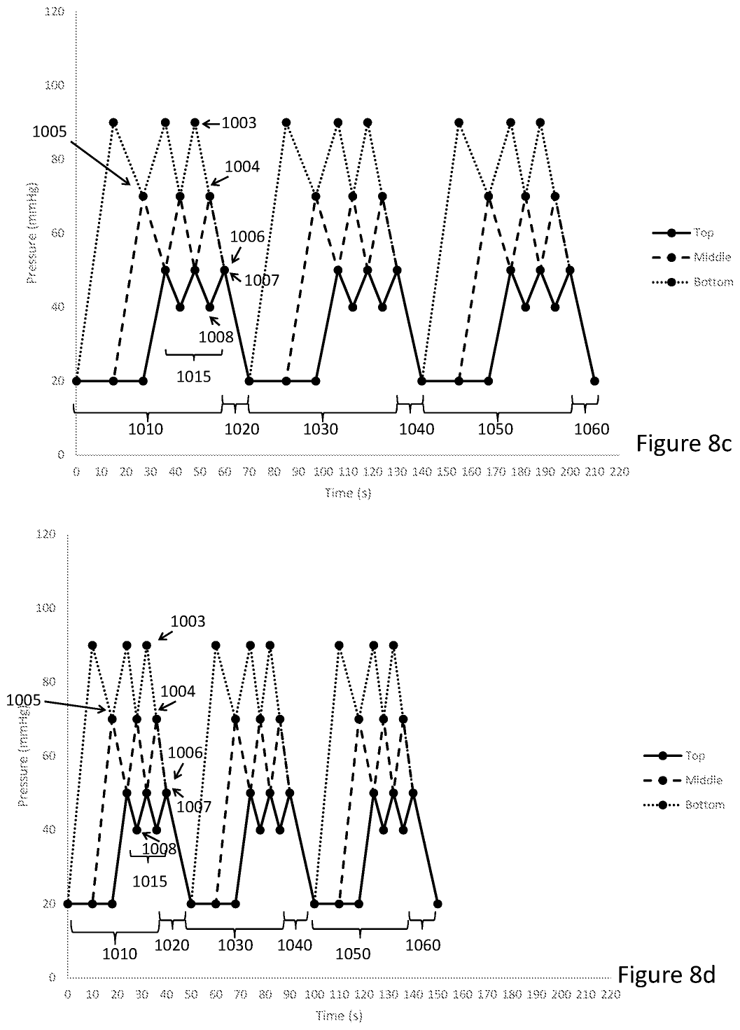

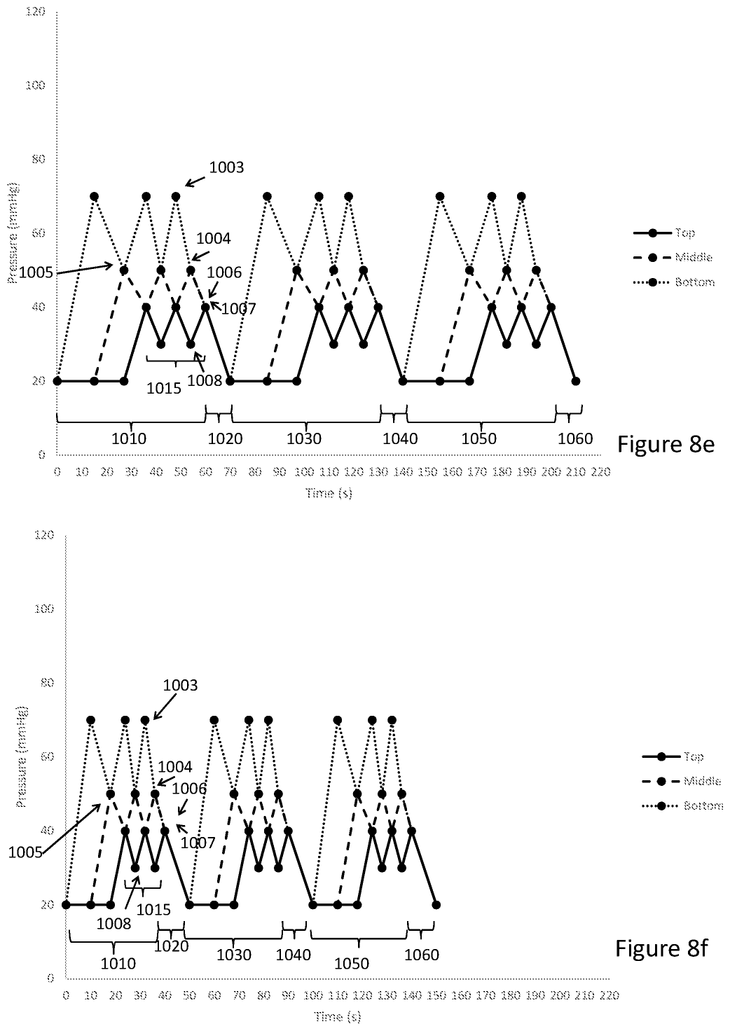

Further embodiments are possible in which the bottom, middle and top peak values are equal and as described above in reference to FIGS. 8a to 8f, the compression in the bottom compression zone starts earlier than the compression in the middle compression zone which in turn starts earlier than the compression in the top compression zone. Thus, the compression pressure applied in the bottom zone is therefore greater than or equal to the compression pressure applied in the middle which in turn is greater than or equal to the compression pressure applied in the top zone.

Further modifications to the values are possible, the compression pressure may take any values in the range 20 mmHg to 200 mmHg. The compression periods may last for between 5 seconds and 5 minutes. The relaxation periods may last for between 5 seconds and 60 seconds. The overall therapy time may last between 5 minutes and 2 hours.

FIGS. 8a to 8f show variations of a second compression pattern type according to an embodiment of the present invention.

The second compression pattern type involves a sequence of compression cycles. Each compression cycle may be considered to comprise a compression period followed by a relaxation period. The compression pressure in each of the compression zones is increased from a initial pressure to a peak pressure value, then the pressure in the zone is decreased to a lower pressure value which is less than the peak pressure value but greater than the initial pressure. Each of the compression periods includes a plurality of mini cycle in which the compression pressure is varied between the peak pressure value and the lower pressure value for that compression zone. Following the compression period, there is a relaxation period. During the relaxation period, the pressure in each of the compression zones returns to the initial pressure.

As shown in FIG. 8a, initially all of the compression zones are set to the initial pressure of 20 mmHg. Then the compression pressure at the bottom compression zone is gradually increased from the initial pressure to bottom compression zone peak value 1003. At approximately the time that the bottom zone reaches the bottom zone peak value 1003, the compression pressure at the middle compression zone is gradually increased from the initial pressure to a middle compression zone peak value 1005. As the compression pressure in the middle compression zone is increased, the compression pressure in the bottom zone is gradually decreased to a bottom compression zone lower value 1004. In the example shown in FIGS. 8a to 8f, the bottom compression zone lower value 1004 is equal to the middle compression zone peak value 1005. The compression pressure at the bottom compression zone reaches the bottom zone lower compression value 1004 at approximately the same time as the middle compression zone reaches the middle compression zone peak value 1005. At approximately the same time, compression in the top compression zone begins and the compression pressure in the top compression zone is gradually increased to a top compression zone peak value 1007. The compression pressure in the middle compression zone decreases to a middle compression zone lower value 1006 and the compression pressure in the bottom compression zone increases back to the bottom compression zone peak value 1003. The compression pressure in the top zone then decreases to a top compression zone lower value 1008. Thus as shown, the compression pressure in each of the zones alternates between a peak value and a lower value. This alternation may be considered as a series of mini-cycles 1015 in which the compression pressure varies out of phase in neighboring compression zones so that when the compression pressure at the bottom compression zone is at the bottom compression zone peak value 1003, the compression pressure at the middle compression pressure is at the middle compression zone lower value 1006 and the compression pressure at the top zone is at the top compression zone peak value 1007. Further in the example shown in FIGS. 8a to 8f, the bottom compression zone lower value 1004 is equal to the middle compression zone peak value 1005. Similarly, the middle compression zone lower value 1006 is equal to the top compression zone peak value 1007.

Following two complete mini-cycles 1015, the compression pressure in each of the compression zones is gradually decreased back to the initial compression. Thus a complete compression cycle may be considered as a compression period 1010 followed by a relaxation period 1020 in which the compression pressure is reduced to the initial compression. The compression period 1010 comprises a plurality of mini-cycles 1015. A second compression period 1030 and a second relaxation period 1040 then, a third compression period 1050 and a third relaxation period 1060 follow.

In the embodiment shown in FIG. 8a, the compression in the middle compression zone starts 15 seconds after compression in the bottom compression zone. Compression in the top compression zone starts 27 seconds after compression in the bottom zone. The bottom compression zone peak value 1003 is 110 mmHg. The bottom compression zone lower value 1004 is 90 mmHg. The middle compression zone peak value 1005 is 90 mmHg. The middle compression zone lower value 1006 is 70 mmHg. The top compression zone peak value 1007 is 70 mmHg. The top compression zone lower value 1008 is 50 mmHg. Each mini-cycle 1015 lasts 12 seconds. The compression period 1010 lasts 60 seconds and the relaxation period 1020 lasts 10 seconds.

In the embodiment shown in FIG. 8b, the compression in the middle compression zone starts 10 seconds after compression in the bottom compression zone. Compression in the top compression zone starts 18 seconds after compression in the bottom zone. The bottom compression zone peak value 1003 is 110 mmHg. The bottom compression zone lower value 1004 is 90 mmHg. The middle compression zone peak value 1005 is 90 mmHg. The middle compression zone lower value 1006 is 70 mmHg. The top compression zone peak value 1007 is 70 mmHg. The top compression zone lower value 1008 is 50 mmHg. Each mini-cycle 1015 lasts 8 seconds. The compression period 1010 lasts 40 seconds and the relaxation period 1020 lasts 10 seconds.

In the embodiment shown in FIG. 8c, the compression in the middle compression zone starts 15 seconds after compression in the bottom compression zone. Compression in the top compression zone starts 27 seconds after compression in the bottom zone. The bottom compression zone peak value 1003 is 90 mmHg. The bottom compression zone lower value 1004 is 70 mmHg. The middle compression zone peak value 1005 is 70 mmHg. The middle compression zone lower value 1006 is 50 mmHg. The top compression zone peak value 1007 is 50 mmHg. The top compression zone lower value 1008 is 40 mmHg. Each mini-cycle 1015 lasts 12 seconds. The compression period 1010 lasts 60 seconds and the relaxation period 1020 lasts 10 seconds.

In the embodiment shown in FIG. 8d, the compression in the middle compression zone starts 10 seconds after compression in the bottom compression zone. Compression in the top compression zone starts 18 seconds after compression in the bottom zone. The bottom compression zone peak value 1003 is 90 mmHg. The bottom compression zone lower value 1004 is 70 mmHg. The middle compression zone peak value 1005 is 70 mmHg. The middle compression zone lower value 1006 is 50 mmHg. The top compression zone peak value 1007 is 50 mmHg. The top compression zone lower value 1008 is 40 mmHg. Each mini-cycle 1015 lasts 8 seconds. The compression period 1010 lasts 40 seconds and the relaxation period 1020 lasts 10 seconds.

In the embodiment shown in FIG. 8e, the compression in the middle compression zone starts 15 seconds after compression in the bottom compression zone. Compression in the top compression zone starts 27 seconds after compression in the bottom zone. The bottom compression zone peak value 1003 is 70 mmHg. The bottom compression zone lower value 1004 is 50 mmHg. The middle compression zone peak value 1005 is 50 mmHg. The middle compression zone lower value 1006 is 40 mmHg. The top compression zone peak value 1007 is 40 mmHg. The top compression zone lower value 1008 is 30 mmHg. Each mini-cycle 1015 lasts 12 seconds. The compression period 1010 lasts 60 seconds and the relaxation period 1020 lasts 10 seconds.

In the embodiment shown in FIG. 8f, the compression in the middle compression zone starts 10 seconds after compression in the bottom compression zone. Compression in the top compression zone starts 18 seconds after compression in the bottom zone. The bottom compression zone peak value 1003 is 70 mmHg. The bottom compression zone lower value 1004 is 50 mmHg. The middle compression zone peak value 1005 is 50 mmHg. The middle compression zone lower value 1006 is 40 mmHg. The top compression zone peak value 1007 is 40 mmHg. The top compression zone lower value 1008 is 30 mmHg. Each mini-cycle 1015 lasts 8 seconds. The compression period 1010 lasts 40 seconds and the relaxation period 1020 lasts 10 seconds.

In addition to the embodiments described above, modifications to the values are possible, the compression pressure may take any values in the range 20 mmHg to 200 mmHg. The compression periods may last for between 5 seconds and 5 minutes. The relaxation periods may last for between 5 seconds and 60 seconds. The overall therapy time may last between 5 minutes and 2 hours.

As described above, in the compression patterns, graduated compression is applied to the different compression zones. The compression patterns may be applied to different parts of the limb and the patterns may be applied to either the arms or legs of a subject. It is noted that there is a pressure gradient in the patterns from a distal part of the limb to a proximal part of the limb. This graduated pressure improves venous return. It is noted that in addition to enhancing blood circulation, the compression devices and compression patterns described herein may also enhance lymph flow in a limb of a subject.

In addition, the compression patterns may be beneficial for subjects such as athletes when recovering. This is because the external pressure reduces intramuscular space available for swelling and promotes the stable alignment of muscle fibers. This reduces the inflammatory response and muscle soreness. This is beneficial in improving psychological aspects of recovery and may have potential benefits for injury prevention and management.

Usage of active compression has been rapidly increasing due to its added benefits compared to static compression.

Static compression which is mostly provided through compression socks reduces valve distension which in turn improves blood flow. Degradation of valves (which causes valve distension) results in venous insufficiency and possibly other conditions such as varicose veins as the blood flow to the heart is reduced due to reflux.

However, static compression assists the user mostly when he/she is mobile as calf muscle pump is inactive when seated.

Added advantage of active compression is that it mimics the calf muscle pump and supports the user even when he/she is seated. Active compression empties lower compartments of the leg preventing blood pooling which in turn increases venous flow towards the heart.

Given the fact that static and active compressions serve two different purposes it is evident that having both components in a compression therapy device is an advantage, thus in some embodiments, the compression devices and compression patterns described above are used in conjunction with a compression sock.

The inner compression sock would hold the valves conjointly whilst the outer active layer sequentially compresses the calf muscle and empties the veins.

The following studies were conducted for the compression pattern described above with reference to FIGS. 7a to 7f.

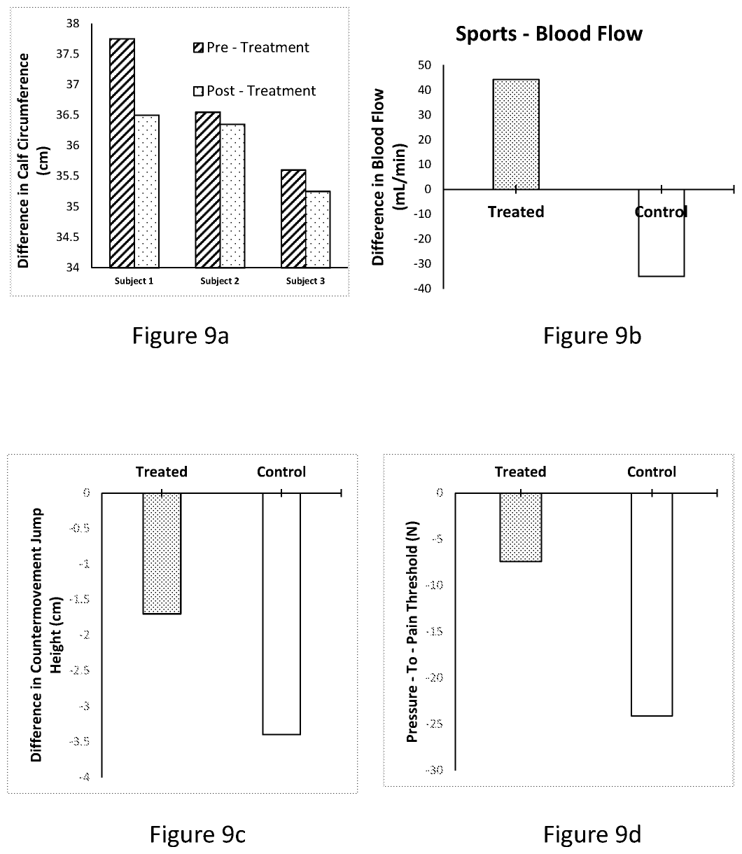

FIGS. 9a to 9d show results of a study done to evaluate the benefit of using a compression device according to an embodiment of the present invention on a group of athletes. A group of subjects (3 males and 3 females) between the age of 19-40 years with no vasoactive or glycemic-control related medications were chosen for this randomized trial. A sub-group of participants (3 subjects) were treated with active compression and the other group was used as the control (with passive rest).

Following parameters were assessed using the respective techniques. The calf circumference was measured using a tape, Femoral arterial blood flow was quantified using a 5-12 MHz multi-frequency linear phase array ultrasound transducer (SonoScape S2, SonoScape Medical Corp, Shenzhen, China), pressure-to-pain threshold (PPT) of the calf was measured using an algometer (to assess muscle soreness) and static vertical jump and countermovement jump tests was performed to assess the improvement in performance.

Following the baseline measurements of the above parameters the participants completed 10 sets of 10 drop jumps from a 0.43 m high box with 10 sec rest between repeats and 1-min rest between sets. Immediately following exercise, muscle soreness was again measured and half of the participants were then treated for 30-min via the active compression device. At 15-min of treatment, femoral artery blood flow was measured. After treatment, all baseline measurements were repeated.

FIG. 9a shows the difference in calf circumference pre-treatment and post-treatment for subjects using active compression. Following the above intense workout protocol and respective treatments, the change in circumference of the calf tended to be smaller with active compression treatment compared to control. On average active compression reduced calf circumference by 0.6 cm, compared to the control group in which the reduction was 0.37 cm. In the treated group, pre-treatment calf circumferences of Subject 1, Subject 2 and Subject 3 were 37.75 cm, 36.55 cm and 35.6 cm, respectively. Following treatment the respective calf circumferences reduced to 36.5 cm, 36.35 cm and 35.25 cm

FIG. 9b shows the difference in femoral arterial blood flow for the group of subjects treated with the compression device and the control group. Femoral artery blood flow was increased relative to baseline during treatment with active compression device and decreased during time-control (Active compression treated: 44.3 mL/min; control group: −35.0 mL/min).

FIG. 9c shows the difference in counter-movement jump height for the subjects treated with the compression device and the control group. Compared to baseline, there was a reduction in counter-movement jump performance for both groups. For the control group this was 3.4 cm. However, this reduction was 1.7 cm in the group treated with active compression. Thus, active compression treatment resulted in 50% improvement in performance compared to the control group.

FIG. 9d shows an indication of soreness for for the subjects treated with the compression device and the control group. Compared to baseline measurements, the active compression treatment was associated with marked reduction in calf soreness following the intense workout protocol and treatment. Active compression: −7.4 N, Control: 24.1 N. It is noted that a decrease in the pressure to pain threshold (PPT) indicates more soreness as less pressure needs to be applied before pain is felt by the participant.

This study has shown that the said active compression technology appears to mitigate calf swelling which occurs due to intense workout procedures, which in this study was mimicked by 100 jumps. Active compression increases blood flow during the treatment and thereby improves performance. The said active compression technology is associated with marked reduction in calf muscle soreness which occurs due to tiring activities.

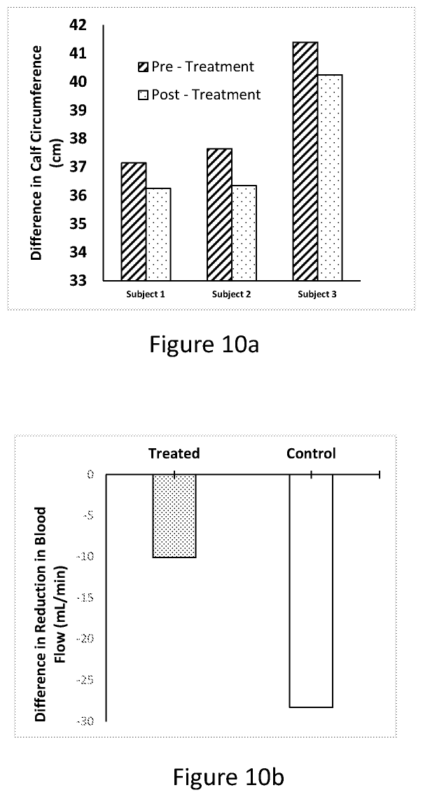

FIGS. 10a and 10b show results of a study done to evaluate the benefit of using a compression device according to an embodiment of the present invention on a group of subjects who were seated for a long period. A group of subjects (1 male and 2 females) between the age of 19-40 years with no vasoactive or glycemic-control related medications were chosen for this crossover trial. Each participant completed 2 protocols (i.e., two conditions). Treatment modalities included active compression and control (i.e., passive rest). The subjects had to report to the trial site on two separate, adjacent days.

Following parameters were assessed using the respective techniques. Calf circumference was measured using a measuring tape and femoral arterial blood flow was quantified using a 5-12 MHz multi-frequency linear phase array ultrasound transducer (SonoScape S2, SonoScape Medical Corp, Shenzhen, China).

The baseline of above measurements was acquired after which the subjects were asked to sit for a prolonged period of time to represent the long distance traveller's condition—upright sitting in a chair with hips and knees at a 90 degree angle for 2 hrs.

The subjects were randomly assigned to either active compression or passive rest on Day 1 (Active compression—continuous cycles of 20-min on, 10-min off during their sitting period). The subjects assigned to active compression on Day 1 received passive rest on Day 2 and vice versa.

FIG. 10a shows the difference in calf circumference pre-treatment and post-treatment for subjects using active compression. As a result of sitting quietly for 2-h, calf circumference increased by 0.57 cm from the baseline measurements when the subjects were not exposed to active compression. When the subjects were treated with Active compression (continuous cycles of 20-min on, 10-min off) for 2-h, calf circumference decreased by 1.11 cm. Pre-treatment calf circumferences of Subject 1, Subject 2 and Subject 3 were 37.15 cm, 37.65 cm and 41.4 cm, respectively. Following treatment the respective calf circumferences reduced to 36.25 cm, 36.35 cm and 40.25 cm

FIG. 10b shows the difference in femoral arterial blood flow for the group of subjects treated with the compression device and the control group. Compared to baseline measurements, femoral artery blood flow was decreased by 28.3 mL/min at 2-h of sitting, when the subjects were not exposed to active compression. However, when the subjects were treated with Active compression (continuous cycles of 20-min on, 10-min off) for 2-h, femoral artery blood flow was decreased only by 10.1 mL/min at 2-h of sitting.

These experiments confirmed the said active compression technology appears to prevent calf swelling which occurs due to long-term sitting and it also acts to mitigate decrease in blood flow.

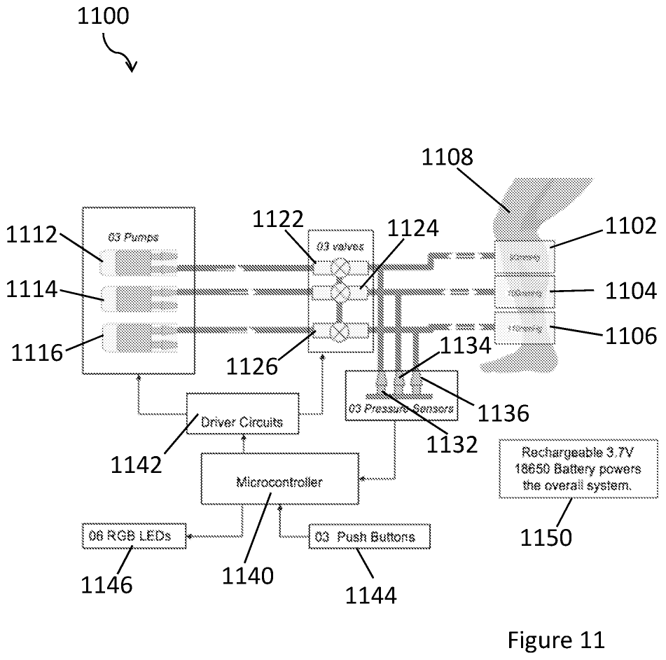

FIG. 11 schematically shows a pneumatic compression device according to an embodiment of the invention. The pneumatic compression device 1100 comprises three inflatable bladders: a top inflatable bladder 1102, a middle inflatable bladder 1104 and a bottom inflatable bladder 1106. As shown in FIG. 11, the three inflatable bladders are arranged to apply compression to top, middle and bottom compression zones respectively of a calf of a wear's leg 1108.

A top compression zone pump 1112 is connected to the top inflatable bladder 1102 via a top compression zone valve 1122. A middle compression zone pump 1114 is connected to the middle inflatable bladder 1104 via a middle compression zone valve 1124. A bottom compression zone pump 1116 is connected to the bottom inflatable bladder 1106 via a bottom compression zone valve 1126. The respective inflatable bladders, valves and pumps are connected by tubing. A top compression zone pressure sensor 1132 is connected to the tubing between the top inflatable bladder 1102 and the top compression zone valve 1122. A middle compression zone pressure sensor 1134 is connected to the tubing between the middle inflatable bladder 1104 and the middle compression zone valve 1124. A bottom compression zone pressure sensor 1136 is connected to the tubing between the bottom inflatable bladder 1106 and the bottom compression zone valve 1126.

A microcontroller 1140 is coupled to driver circuits 1142 which allow individual control of the top compression zone pump 1112, the middle compression zone pump 1114 and the bottom compression zone pump 1116, and individual control of the top compression zone valve 1122, the middle compression zone valve 1124 and the bottom compression zone valve 1126. The microcontroller 1140 is coupled to the A top compression zone pressure sensor 1132, the middle compression zone pressure sensor 1134 and the bottom compression zone pressure sensor 1136.

A set of three push buttons 1144 allow user input of commands into the pneumatic compression device 1100. A set of 6 RGB (red, green, blue) light emitting diodes (LEDs) 1146 display information about the selected mode of the pneumatic compression device 1100. Power is supplied to the pneumatic compression device 1100 by a battery 1150.

In use, the pumps are controlled by the microcontroller 1140 to inflate respective individual inflatable bladders according to a compression pattern such as those described above with reference to FIGS. 7a-f and 8a-f. The pressure sensors provide feedback to the microcontroller 1140 so that the inflatable bladders can individually be inflated to a required pressure. In order to hold individual inflatable bladders at a desired pressure, the corresponding valve is closed. To release the pressure in one of the inflatable bladders, the corresponding valve is opened with allows air to flow out of the bladder and through the corresponding pump.

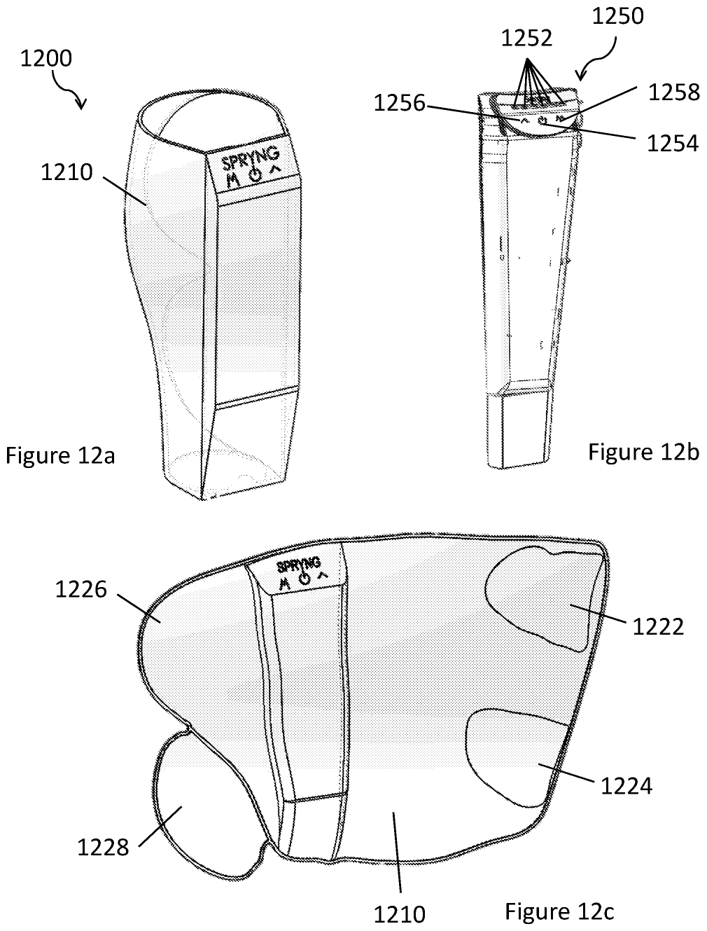

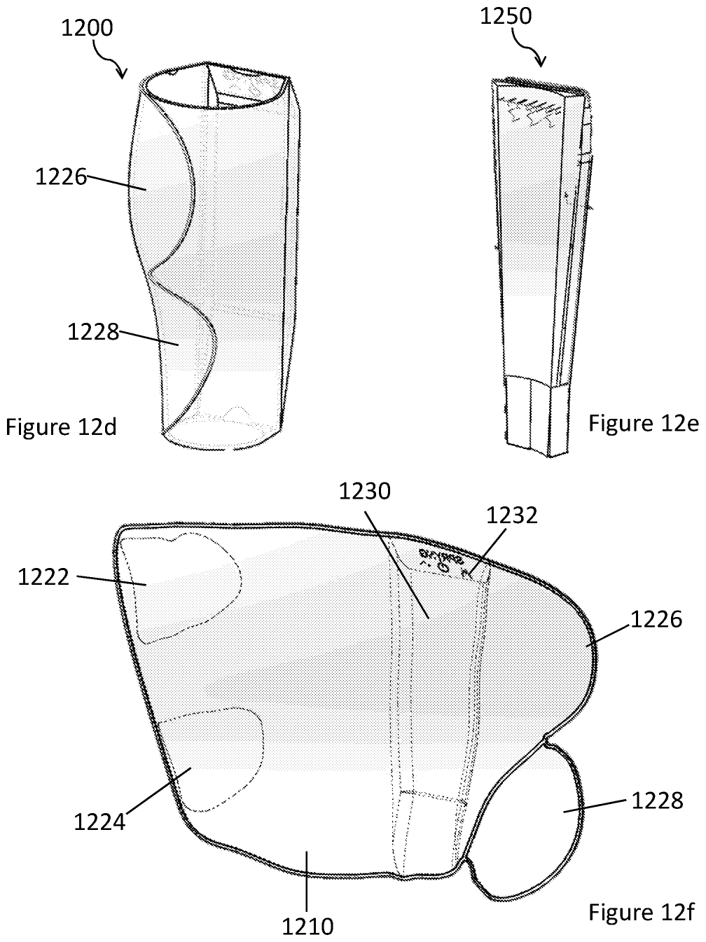

FIGS. 12a to 12f show a pneumatic compression device according to an embodiment of the present invention. FIG. 12a is a front view of the pneumatic compression device in a closed configuration. FIG. 12b is a front view of an actuator portion of the pneumatic compression device. FIG. 12c is s front view of the pneumatic compression device in an open configuration. FIG. 12d is a rear view of the pneumatic compression device in a closed configuration. FIG. 12e is a rear view of the actuator portion of the pneumatic compression device. FIG. 12f is a rear view of the pneumatic compression device in an open configuration.

As shown in FIGS. 12a to 12f, the pneumatic compression device 1200 comprises flexible portion or active layer 1210 which is in the form of a sleeve that is wrapped around lower leg of the user, and an actuator portion 1250 which is accommodated in a pocket in the front of the body portion 1210. When the compression device 1200 is worn, the actuator portion 1250 is located over the shin of the wear. The actuator portion 1250 has six LEDs 1252 which correspond to LEDs 1146 shown in FIG. 11 located at the top end. Below the LEDs 1252 are three control buttons: a power on/off button 1254; a compression intensity selection button 1256 and a compression pattern selection button 1258 which correspond to the push buttons 1144 shown in FIG. 11.

The active layer 1210 comprises an upper fastening portion 1222 and a lower fastening portion 1224 and an upper fastening tab 1126 on one side and a lower fastening tab 1128 on the other side. The upper fastening portion 1222, the lower fastening portion 1124, the upper fastening tab 1226 and the lower fastening tab 1128 comprise a fastening surface such as Velcro which allow the pneumatic compression garment to be fastened around the lower leg of the wearer.

The active layer 1210 comprises a pocket 1230 into which the actuator portion 1250 is inserted. The top of the pocket 1230 has labels corresponding to the control buttons of the actuator portion 1250.

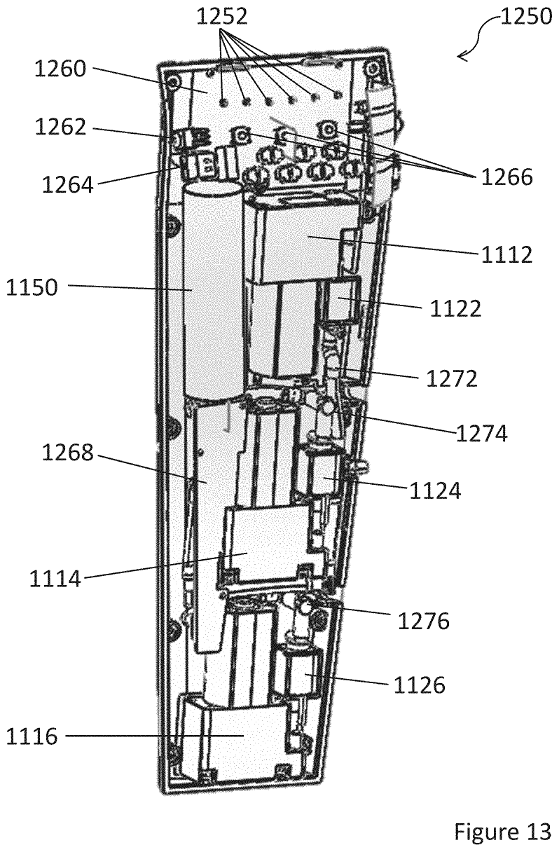

FIG. 13 shows the internal configuration of an actuator portion of a pneumatic compression device according to an embodiment of the present invention. FIG. 13 is a view from the back of the actuator portion 1250 with the back cover (which faces the shin of the wearer when the actuator device is in use) removed.

As shown in FIG. 13, a first printed circuit board (PCB) 1260 is located at the top of the actuator portion 1250. Six LEDs 1252 and three buttons 1266 are arranged on the PCB 1260. At the side of the PCB 1260 an infrared communication module 1262 and a universal serial bus (USB) charging port 1264 are mounted. It is noted that the infrared communication module 1262 is located on the left hand side of the actuator portion 1250 as shown in FIG. 13. When two compression devices are used together with one on each leg of a wearer, the infrared communication modules of the two compression devices are arranged to face one another. Thus, the actuator portion 1250 shown in FIG. 13 corresponds to a right leg compression device and a corresponding left leg compression device would have the infrared communication module arranged at the opposite side.

The battery 1150 is located below the first PCB 1260. The top compression zone pump 1112 and the top compression zone valve 1122 are located laterally from the battery 1150. A top compression zone tube connector 1272 is connects to tubing from the top compression zone pump 1112. A second PCB 1268 is located below the battery 1150 and comprises circuitry associated with the pressure sensors. The middle compression zone pump 1114 and the middle compression zone valve are located laterally from the second PCB 1268. A middle compression zone connector 1274 connects to tubing from the middle compression zone pump 1114. The bottom compression zone pump 1116 and the bottom compression zone valve 1126 are located below the middle compression zone pump 1114. A bottom compression zone connector 1276 connects to tubing from the bottom compression zone pump 1116.

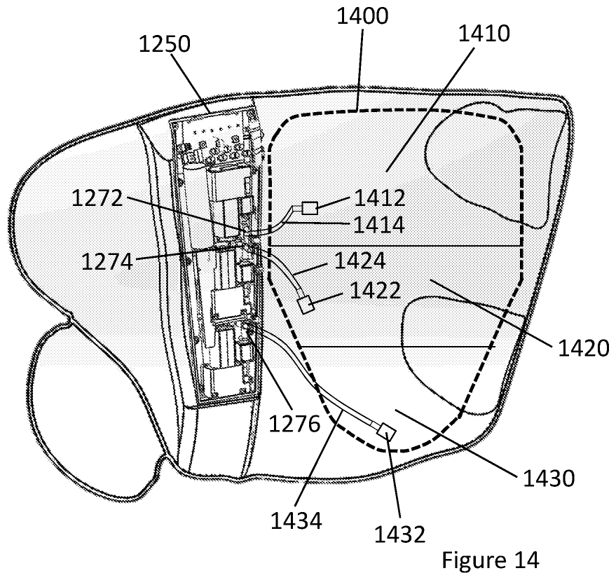

FIG. 14 shows an inflatable bladder component within a pneumatic compression device according to an embodiment of the present invention. The inflatable bladder component 1400 is located within a pocket in the active layer 1210 of the pneumatic compression device. The inflatable bladder component 1400 comprises three inflatable bladders: a top inflatable bladder 1410, a middle inflatable bladder 1420 and a bottom inflatable bladder 1430. The top inflatable bladder 1410 has a top bladder port 1412 which is connected to a top bladder connecting tube 1414. The top bladder connecting tube 1414 connects the top inflatable bladder 1410 to the top compression zone tube connector 1272 of the actuator module 1250. The middle inflatable bladder 1420 has a middle bladder port 1422 which is connected to a middle bladder connecting tube 1424. The middle bladder connecting tube 1424 connects the middle inflatable bladder 1420 to the middle compression zone connector 1274 of the actuator module 1250. The bottom inflatable bladder 1430 has a bottom bladder port 1432 which is connected to a bottom bladder connecting tube 1434. The bottom bladder connecting tube 1432 connects the bottom inflatable bladder 1430 to the bottom compression zone connector 1276 of the actuator module 1250.

When the compression device 1200 is worn, the three inflatable bladders are arranged against calf muscle of the wearer. By inflating the inflatable bladders, compression patterns can be applied to the wearer's calf.

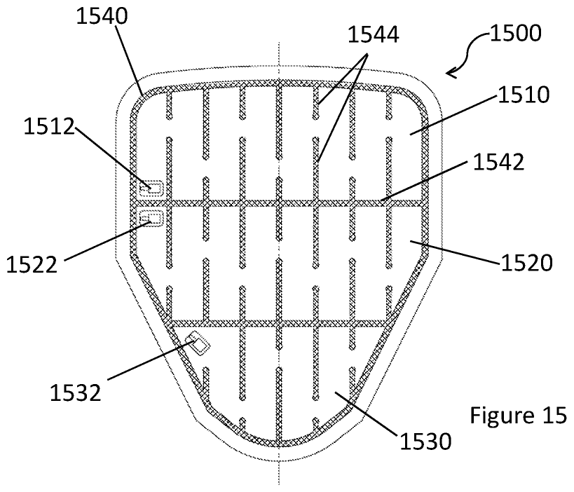

FIG. 15 shows an inflatable bladder module of a compression device according to an embodiment of the present invention. The inflatable bladder module 1500 is formed from two membranes formed from sheets of Thermoplastic polyurethane (TPU) which are bonded together along bonding lines. For example, heat bonding may be used to bond the two sheets together. The top inflatable bladder 1510, the middle inflatable bladder 1520 and the bottom inflatable bladder 1530 are formed by an exterior boundary 1540 which is formed from a bond that runs around the circumference of the inflatable bladder module 1500. Two internal boundaries 1542 separate the top inflatable bladder 1510 from the middle inflatable bladder 1520, and the middle inflatable bladder 1520 from the bottom inflatable bladder 1530, respectively. In this embodiment, the internal boundaries 1542 are substantially straight and run in a horizontal direction.

The top bladder port 1512, the middle bladder port 1522 and the bottom bladder port 1532 located close to the exterior boundary 1540 in the respective inflatable bladders.

As shown in FIG. 15, a plurality of baffles 1544 are located within each of the inflatable bladders. The baffles 1544 are formed as bonds between the membranes that form the inflatable bladder module 1500. The baffles 1544 run in a vertical direction which is along the axis of the leg of the wearer of the compression device. The baffles 1544 form a set of parallel lines with breaks that are offset on neighboring baffles.

The baffles 1544 prevent the inflatable bladders from bulging excessively. The arrangement of baffles in the vertical direction allows the bladder component 1500 to bend around the leg of the wearer.

In some embodiments, a pair of compression devices are each provided with a wireless communication module to allow synchronization of compression patterns applied to each leg of a wearer.

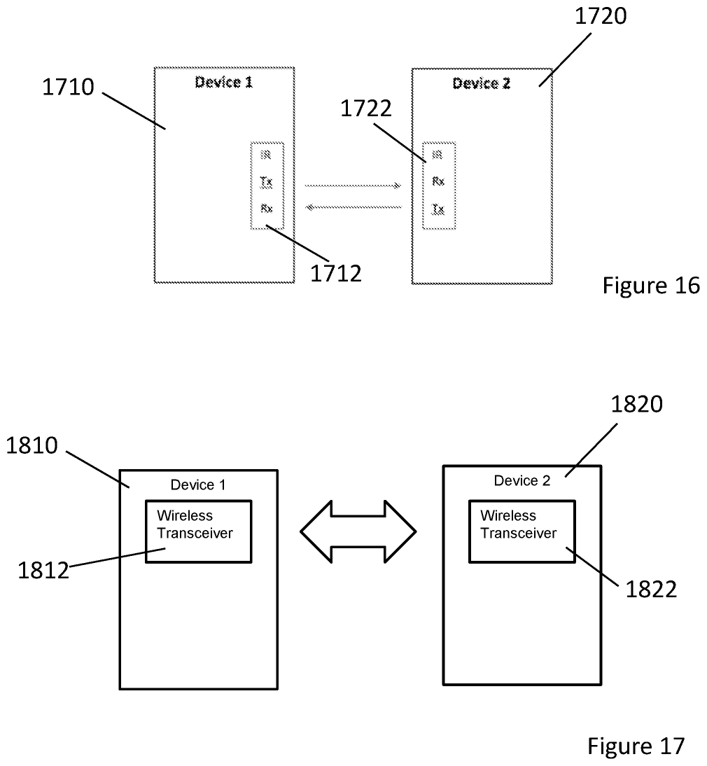

FIG. 16 shows a pair of compression devices which are provided with infrared communication modules according to an embodiment of the present invention. As shown in FIG. 16, a first compression device 1710 comprises a first infrared communication module 1712 and a second compression device 1720 comprises a second infrared communication module 1722.

In this example embodiment, the first compression device is configured as a master device and the second compression device is configured as a slave device. The master compression device comprises three buttons: a power on/off button, an intensity selection button and a mode selection button. In this example, there are two possible modes which may be selected and three possible intensities for each mode. Table 1 below shows the signals generated by the infrared communication module 1712 of the master device (the first compression device 1710) and the synchronization carried out by the slave device (the second compression device 1720) in response to receiving an infrared signal at the second infrared communication module 1722.

TABLE 1

| | Signal Generated | |
|---|---|---|
| Button Pressed | by Master | Synchronization by Slave |
| in Master Device | through IR | Device |
| Power On | 0 | The slave restarts the default |
| | | therapy & intensity as soon as it |
| | | receives a “0” through IR. |
| | | This action syncs the compression |
| | | pattern with Master. |
| Resume (Power | 1 | The slave resumes to therapy as |
| Button- Short | | soon as it receives a “1” through |
| Press) | | IR |
| Pause (Power | 2 | The slave pauses the therapy as |
| Button- Short | | soon as it receives a “2” through |
| Press) | | IR |
| Mode (choose | 3 | The slave begins the compression |
| Mode 1 when | | therapy in Mode 1 & Intensity 1 as |
| Intensity 1) | | soon as it receives a “3” through |
| | | IR |
| Mode (choose | 4 | The slave begins the compression |
| Mode 1 when | | therapy in Mode 1 & Intensity 2 as |
| Intensity 2) | | soon as it receives a “4” through |
| | | IR |
| Mode (choose | 5 | The slave begins the compression |
| Mode 1 when | | therapy in Mode 1 & Intensity 3 as |
| Intensity 3) | | soon as it receives a “5” through |
| | | IR |
| Mode (choose | 6 | The slave begins the compression |
| Mode 2 when | | therapy in Mode 2 & Intensity 1 as |
| Intensity 1) | | soon as it receives a “6” through |
| | | IR |
| Mode (choose | 7 | The slave begins the compression |
| Mode 2 when | | therapy in Mode 2 & Intensity 2 as |
| Intensity 2) | | soon as it receives a “7” through |
| | | IR |
| Mode (choose | 8 | The slave begins the compression |
| Mode 2 when | | therapy in Mode 2 & Intensity 3 as |
| Intensity 3) | | soon as it receives a “8” through |
| | | IR |
| Power off | 9 | The slave will power off as soon as |
| | | it receives a “9” through IR |

As shown in table 1 above, in response to an input on the master device, the slave device will perform a corresponding action following receipt of an infrared signal.

FIG. 17 shows a pair of compression devices which are provided with wireless communication modules. As shown in FIG. 17, the first compression device 1810 comprises a first wireless transceiver 1812 and the second compression device 1820 comprises a second wireless transceiver 1822. The first and second wireless transceivers may be Bluetooth low energy (BLE) transceivers.

In this embodiment the two compression devices communicate with each other using Bluetooth Low Energy (BLE) communication technology to ensure the compression sequence in both devices are time, pattern & intensity synchronized. Each device in a pair has a BLE transceiver unit which can send and receive BLE signals which contains mode, intensity, pause, resume related data encoded in it. The data in BLE signal is decoded by the receiving unit and processed to implement synchronization between the sending device and receiving device.

The first compression device 1810 is configured as a Master and the second communication device 1820 is configured as a Slave. Both the devices (Master & Slave) must be powered on independently. Once the devices are powered on, the user can select the Mode and/or intensity by pressing Mode & Intensity buttons on the master device. The BLE transceiver inside the Master device will transmit a data packet to the Slave device when one of the User Interface buttons (Mode, Intensity & Pause) are pressed. The data packet will include information about the Mode, Intensity selected in the Master device. The Slave device will receive the data packet instantaneously and will use the information in the data packet to set the Mode and Intensity of the Slave device—this way, both the devices will be time, mode & intensity synched.

The Master device and Slave device doesn't need to aligned to face each other for successful communication between them. They both have to within a range of 5 m to have successful communication and they have to be “paired” prior to successful communication. This pairing will done at factory during mass production.

The table shows the BLE signals sent by Master Device to the Slave device based on button press. When these BLE signals are received by the Slave, it will choose a state (Pause, Resume, Mode, Intensity, Power-off) from the pre-programmed look-up table based on the received BLE signal (1, 2, 3 . . . 9) from the Master.

TABLE 2

| | Signal Generated | |
|---|---|---|
| Button Pressed | by Master | Synchronization by Slave |
| in Master Device | through BLE | Device |
| Power On | 0 | The slave restarts the default |
| | | therapy & intensity as soon as it |
| | | receives a “0” through BLE. |
| | | This action syncs the compression |
| | | pattern with Master. |
| Resume (Power | 1 | The slave resumes to therapy as |
| Button- Short | | soon as it receives a “1” through |
| Press) | | BLE |
| Pause (Power | 2 | The slave pauses the therapy as |
| Button- Short | | soon as it receives a “2” through |
| Press) | | BLE |
| Mode (choose | 3 | The slave begins the compression |
| Mode 1 when | | therapy in Mode 1 & Intensity 1 as |
| Intensity 1) | | soon as it receives a “3” through |
| | | BLE |
| Mode (choose | 4 | The slave begins the compression |
| Mode 1 when | | therapy in Mode 1 & Intensity 2 as |
| Intensity 2) | | soon as it receives a “4” through |
| | | BLE |
| Mode (choose | 5 | The slave begins the compression |
| Mode 1 when | | therapy in Mode 1 & Intensity 3 as |
| Intensity 3) | | soon as it receives a “5” through |
| | | BLE |
| Mode (choose | 6 | The slave begins the compression |
| Mode 2 when | | therapy in Mode 2 & Intensity 1 as |
| Intensity 1) | | soon as it receives a “6” through |
| | | BLE |
| Mode (choose | 7 | The slave begins the compression |
| Mode 2 when | | therapy in Mode 2 & Intensity 2 as |
| Intensity 2) | | soon as it receives a “7” through |
| | | BLE |
| Mode (choose | 8 | The slave begins the compression |
| Mode 2 when | | therapy in Mode 2 & Intensity 3 as |
| Intensity 3) | | soon as it receives a “8” through |
| | | BLE |
| Power off | 9 | The slave will power off as soon as |
| | | it receives a “9” through BLE |

Whilst the foregoing description has described exemplary embodiments, it will be understood by those skilled in the art that many variations of the embodiments can be made within the scope and spirit of the present invention.

1. A method of controlling a compression device, the compression device being configured to apply compression to a plurality of compression zones on a limb of a subject, the compression zones each being a different distance from the torso of the subject, the method comprising:
in a compression period, generating control signals to separately vary the pressure applied in each of the compression zones such that for each adjacent pair of compression zones the pressure applied at a distal zone of the pair of compression zones is greater than or equal to the pressure applied at a proximal zone of the pair of compression zones,
wherein in at least part of the compression period, the pressure in each of the compression zones is varied simultaneously.

2. (canceled)

3. A method according to claim 1, comprising generating control signals for a plurality of compression cycles, each compression cycle comprising a compression period followed by a relaxation period.

4. A method according to claim 3, comprising generating control signals to relax the pressure applied in each compression zone during the relaxation period.

5. A method according to claim 1, wherein during the compression period, the pressure applied in each of the zones is gradually increased from a first pressure to a second pressure and compression of the distal zone of a pair of compression zones starts before the proximal zone of the pair of zones.

6. A method according to claim 1, wherein the compression pressure applied to each compression zone is repeatedly varied between a maximum value for that zone and a minimum value for each zone.

7. A method according to claim 6, wherein the maximum value for the proximal zone of the pair of zones is substantially equal to the minimum value for the distal zone of the pair of zones.

8. A method according claim 6 wherein the difference between the maximum value and the minimum value for a zone is less than 30 mmHg and preferably less than 20 mmHg.

9. A method according claim 1, further comprising sending a synchronization signal to a second compression device.

10. A method according to claim 9, wherein the synchronization signal is an infrared signal or a wireless network signal.

11. A method according to claim 9, wherein the synchronization signal comprises an indication of a compression pattern.

12. A compression device configured to apply compression to a plurality of compression zones on a limb of a subject, the compression zones each being a different distance from the torso of the subject, the compression device comprising a controller configured to;
generate control signals to separately vary the pressure applied in each of the compression zones, in a compression period, such that for each adjacent pair of compression zones the pressure applied at a distal zone of the pair of compression zones is greater than or equal to the pressure applied at a proximal zone of the pair of compression zones,
wherein in at least part of the compression period, the pressure in each of the compression zones is varied simultaneously.

13. A compression device according to claim 12, further comprising a communication module configured to send a synchronization signal to a second compression device, receive a synchronization signal from a second compression device, or combinations thereof.

14. A computer readable carrier medium carrying instructions which are executable by a controller of a compression device to cause the device to operate according to the method of claim 1.

15. A method of enhancing blood and/or lymph flow in a limb of a subject, the method comprising applying a compression device configured to apply compression to a plurality of compression zones to a limb of a subject and controlling the compression device according to the method of claim 1.

16. A method of aiding recovery from physical exertion in a subject, the method comprising applying a compression device configured to apply compression to a plurality of compression zones to a limb of a subject and controlling the compression device according to the method of claim 1.

17. A method according to claim 15, comprising applying a compression sleeve to the limb of the subject under the compression device.

18.-30. (canceled)

31. A compression device according to claim 12, the controller being further configured to: generate control signals for a plurality of compression cycles, each compression cycle comprising a compression period followed by a relaxation period.

32. A compression device according to claim 31, the controller being further configured to: generate control signals to relax the pressure applied in each compression zone during the relaxation period.

33. A compression device according to claim 12, the controller being further configured to: generate control signals such that during compression period, the pressure applied in each of the zones is gradually increased from a first pressure to a second pressure and compression of the distal zone of a pair of compression zones starts before the proximal zone of the pair of zones.

34. A compression device according to claim 12, the controller being further configured to: generate control signals such that the compression pressure applied to each compression zone is repeatedly varied between a maximum value for that zone and a minimum value for each zone.
